(19) United States
(12) Patent Application Publication
(10) Pub. No.: US 2024/0358600 A1
(43) Pub. Date: Oct. 31, 2024

(54) A Gastrojejunostomy Tube Enabling Bedside Exchange Without Radiation, Sedation, or Procedure Room Time

(71) Applicant: THE JOHNS HOPKINS UNIVERSITY, Baltimore, MD (US)

(72) Inventors: Andrew Demmert, Elkridge, MD (US); Clifford R Weiss, Baltimore, MD (US); Christopher Ravi Bailey, Baltimore, MD (US); Aryaman Gupta, Zionsville, IN (US)

(21) Appl. No.: 18/556,244

(22) PCT Filed: Apr. 19, 2022
(86) PCT No.: PCT/US2022/025380

§ 371 Date: Oct. 19, 2023

Related U.S. Application Data
(60) Provisional application No. 63/176,486, filed on Apr. 19, 2021.

(51) Int. Cl. A61J 15/00; A61M 25/00; A61M 25/06; A61M 25/10

(52) U.S. Cl. CPC A61J 15/0069; A61J 15/0023; A61J 15/0042; A61J 15/0061; A61M 2025/0047; A61M 25/0097; A61M 2025/0681; A61M 25/10

(57) ABSTRACT

A sleeved gastrojejunostomy (GJ) tube, according to the present invention, includes an inner cannula or feeding cannula that is housed in a sleeve. This arrangement allows the inner cannula to be replaced easily at the bedside by non-specialists. Traditional methods to insert the sleeved GJ tube are utilized, but in the event of feeding cannula damage or clogs, the inner cannula can be easily removed and a new one can be placed, without the need for fluoroscopy or anesthesia. The use of sleeved GJ tubes will expedite patient care, reduce complications, and decrease healthcare costs. The present invention aims to solve the common issue of a clogged GJ Tube. Clogged GJ tubes lead to emergency room visits, hospital admission, and interventional procedures. The present invention would enable exchanges of a clogged feeding cannula by providers or parents without radiation, sedation, or room time.

### CROSS REFERENCE TO RELATED APPLICATIONS

This application claims the benefit of U.S. Provisional Patent Application No. 63/176,486 filed on Apr. 19, 2021, which is incorporated by reference, herein, in its entirety.

### FIELD OF THE INVENTION

The present invention relates generally to medical devices. More particularly the present invention relates to a gastrojejunostomy tube enabling bedside exchange without radiation, sedation, or procedure room time.

### BACKGROUND OF THE INVENTION

Gastrojejunostomy (GJ) tubes enable patients who cannot eat to receive food and medication into their stomach and/or small bowel. When these tubes clog or break, they currently must be exchanged in a procedure suite involving radiation, sedation/anesthesia, and room time. Further, an expensive team including nurses, technologists, Interventional Radiologists, and Anesthesiologists needs to be mobilized. While most exchanges are simple procedures, exchanging a clogged tube can take hours if the physician cannot unclog the tube during the procedure, as the physician must then remove the tube and re-navigate the pylorus and duodenal sweep.

It would therefore be advantageous to provide a gastrojejunostomy tube enabling bedside exchange without radiation, sedation, or procedure room time.

### SUMMARY OF THE INVENTION

In accordance with an embodiment, the present invention provides a sleeved gastrojejunostomy (GJ) tube device an outer sheath having a proximal end and a distal end. An outer wall of the outer sheath defines an inner lumen that extends from the proximal end of the outer sheath to the distal end of the outer sheath. The device includes an inner cannula having a proximal end and a distal end. An outer wall of the inner cannula defines an inner lumen that extends from the proximal end of the inner cannula to the distal end of the inner cannula. The inner cannula is removably disposed within the lumen of the outer sheath. The device also includes a feeding hub disposed at a proximal end of the device. The feeding hub is configured to provide nutrients and medications to a patient.

In accordance with another aspect of the present invention, the device includes a retention mechanism to hold the GJ tube device in place. The retention mechanism can take the form of a balloon. The outer sheath and the inner cannula define fenestrations for delivering medication or other substances to a patient. The device includes a stiffener. The device of claim 1 further includes distal weights positioned on a distal end of the outer sheath. The device includes a locking mechanism to releasably couple the inner cannula to the outer sheath. The locking mechanism can take the form of a threaded tube lock. The device can also include a grommet to cover a stoma of the patient. At least a portion of the inner cannula defines a first lumen and a second lumen.

In accordance with yet another aspect of the present invention, the hub is further configured with a balloon port to allow for insufflation of the balloon. The hub can also include a lubrication port to provide lubrication between the outer sheath and the inner cannula. The device can include silicone, oil-based, or MED6670 and MS3130 lubricants.

In accordance with still another aspect of the present invention, the outer sheath and the inner cannula are formed from silicone. A commercial silicone extruder can be used to form the outer sheath and the inner cannula. Injection molding and casting can also be used. The stiffener is formed from a shape memory metal, a hardened strip of plastic, a hardened strip of rubber, or a hardened integral portion of the outer sheath.

In accordance with another aspect of the present invention, a kit includes the device of described herein. A second kit can include least one replacement inner cannula wherein the replacement inner cannula is configured to replace the inner cannula provided in the kit. The kit can further include a lubricant.

In accordance with another aspect of the present invention, a method of using the device of the present invention allows for the inner cannula to be exchanged outside of a procedure suite. The method can also include removing the inner cannula and delivering nutrition through the outer sheath, as an emergency backup.

### BRIEF DESCRIPTION OF THE FIGURES

FIG. 1 illustrates a side view of a gastrojejunal feeding tube according to the prior art.

FIG. 2 illustrates a side view of a sleeved GJ tube, according to an embodiment of the present invention.

FIG. 3 illustrates a side view of a sleeved GJ tube of the present invention disposed in the intestinal tract.

FIGS. 4A and 4B illustrate sectional and side views of the external portion of the sleeved GJ tube, according to an embodiment of the present invention.

FIGS. 5A and 5B illustrate sectional and side views of the balloon retention mechanism and the fenestrations of the outer sheath, according to an embodiment of the present invention.

FIGS. 6A and 6B illustrate sectional and side views of the stiffener of the outer sheath, according to an embodiment of the present invention.

FIGS. 7A and 7B illustrate sectional and side views of the distal end of the outer sheath along with the distal end of the inner cannula, according to an embodiment of the present invention.

FIGS. 8A and 8B illustrate perspective views of a sleeved GJ tube, according to an embodiment of the present invention.

FIG. 9 illustrates a semi-exploded view of a sleeved GJ tube, according to an embodiment of the present invention.

FIGS. 10A-10D illustrate views of a hub of a sleeved GJ tube, according to an embodiment of the present invention.

FIG. 11 illustrates a sectional view of a lumen arrangement of a device according to the prior art.

FIGS. 12 and 13 illustrate sectional views of a lumen arrangement of a sleeved GJ tube, according to an embodiment of the present invention.

FIG. 14 illustrates a perspective view of a balloon retention mechanism of a sleeved GJ tube, according to an embodiment of the present invention.

FIGS. 15 and 16 illustrate graphical views for the materials tested and lubricants tested, according to embodiments of the present invention.

FIG. 17 illustrates a flow diagram of a patient care flow according to an embodiment of the present invention.

### DETAILED DESCRIPTION OF THE PREFERRED EMBODIMENTS

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the appended claims.

The present invention is directed to a sleeved gastrojejunostomy (GJ) tube, which includes an inner cannula or feeding cannula that is housed in a sleeve. This arrangement allows the inner cannula or feeding cannula to be replaced easily at the bedside by non-specialists. Traditional methods to insert the sleeved GJ tube are utilized, but in the event of feeding cannula damage or clogs, the inner cannula can be easily removed and a new one can be placed, without the need for fluoroscopy or anesthesia. The use of a sleeved GJ tubes, according to the present invention, will expedite patient care, reduce complications, and decrease healthcare costs. A device according to the present invention aims to solve the common issue of a clogged GJ Tube. Clogged GJ tubes lead to emergency room visits, hospital admission, and interventional procedures. The present invention would enable exchanges of a clogged feeding cannula by providers or parents without radiation, sedation, or room time.

The present invention relieves the financial, emotional, and physical stress on the patient and hospital by ‘taking the procedure home’. The high volume of tube replacement at just a handful of hospitals indicates the scope of this problem and the strong need for the solution of the present invention, as illustrated in Table 1.

TABLE 1
Tube replacement estimates per year from local hospitals

| | GJ Tube | | |
|---|---|---|---|
| | Replacements per | | |
| Hospital | Year | Adults | Pediatric |
| Johns Hopkins | 750 | 570 | 180 |
| Hospital | | | |
| Beth Israel Medical | 375 | 375 | — |
| Center | | | |
| Children's National | 400 | — | 400 |
| DC | | | |
| Children's Hospital | 1200 | — | 1200 |
| of Philadelphia | | | |
| Nemours/DuPont | 450 | — | 450 |
| Children's Hospital | | | |
| Delaware | | | |

Table 2 shows the costs associated with the standard of care versus with the present invention (Shuriken Medical).

TABLE 2
Hospital Costs and Savings Per Patient Per Year

| Item | Standard of Care | Shuriken Medical |
|---|---|---|
| GJ Kit Costs | $1,000-$1,500/yr | $1,400-$1,800/yr |
| | (4-6 full kit exchanges | (1-2 full kit + 5 bedside |
| | per year) | inner cannula exchanges |
| | | per year) |
| Hospital Visit Costs | $23,760-$35,640/yr | $5,940-$11,880/yr |
| Indirect Costs | Radiation Exposure | Training for exchange |
| | Caregiver Hardship | |
| | Provider Time | |
| Expert Clinician Time | 750 hours (all patients) | 375 hours (all patients) |
| Total Costs | $24,760-$37,140++ | $7,340-$13,680 |

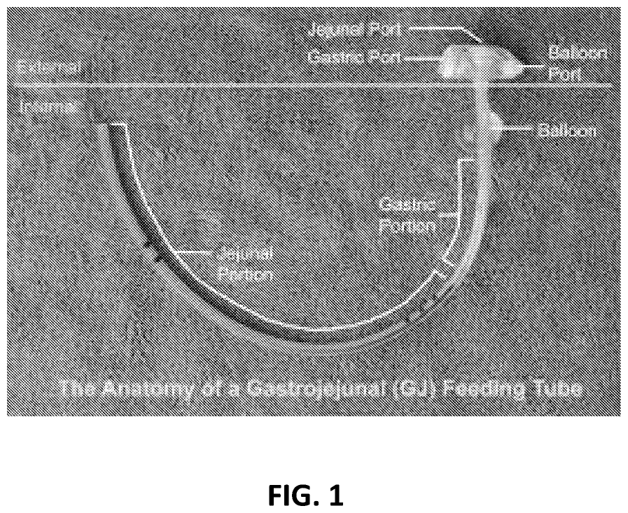

FIG. 1 illustrates a side view of a gastrojejunal feeding tube according to the prior art. The prior art tube includes both internal and external portions. The external portion includes jejunal and gastric ports, as well as a balloon port. The internal portion includes a gastric portion, and a jejunal portion. The internal portion also includes a balloon to secure the gastrojejunal feeding tube in place. The gastrojejunal feeding tube of the prior art is a one piece tube system.

In contrast, the device of the present invention is a two piece tube system in which the inner feeding cannula can be exchanged at home or at bedside without requiring a procedure. The design of the present invention includes several features to decrease tube malfunctions (malposition, etc.):
Weighting the distal tip to help maintain position in the small bowel and decrease the chance of tube malposition or coiling.
Moving the stiffener from inside the tube to the outer sheath, enabling a larger internal lumen diameter for the same outer tube diameter.
Increasing lumen diameter and reducing sharp corners within the lumens to decrease the chance of clogging.
Securing the outer sheath's position with a balloon, a plastic retention device, or other method as diagrammed, as the design allows for a more permanent outer sheath.

The present invention eliminates the need for anesthesia, radiation, and operative equipment. The present invention includes a longer-lasting outer sheath that acts as a track and a replaceable inner tube that can be exchanged at the bedside as needed. The outer sheath has a stiffening element (e.g., NiTiNol), distal weight, retention device (balloon), and ports for gastric feeding and balloon inflation. The inner tube locks at the feeding hub outside the body and extends into the small intestine for intestinal feeding. Currently, no products exist that enable non-specialists to safely replace GJ tubes at the bedside or in the outpatient setting. None of the innovations in the present invention indicate that the procedure will be less successful or more poorly tolerated than the current standard of care. These features are described in further detail with respect to the figures that follow.

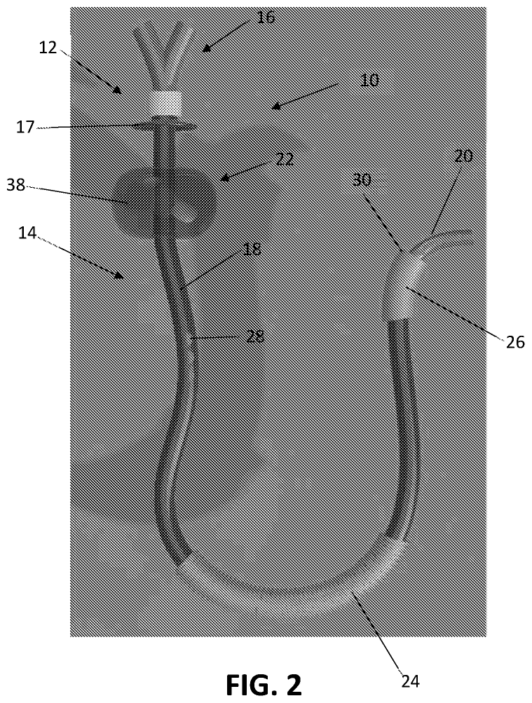

FIG. 2 illustrates a side view of a sleeved GJ tube, according to an embodiment of the present invention. The sleeved GJ tube 10 includes external and internal portions 12, 14. The external portion 12 includes a two-port feeding hub 16. While a two-port feeding hub 16 is shown here, it is to be understood that this is simply included as an example and is not meant to be considered limiting. Any feeding hub configuration known to or conceivable to one of skill in the art could be used. A flange 17 disposed on the external portion 12 covers the stoma of the patient. The inner portion 14 includes an outer sheath 18 and an inner cannula 20. The inner cannula 20 in many embodiments takes the form of a feeding cannula. The inner cannula 20 can be configured to lock with the outer sheath 18 such that unwanted movement between the two components is minimized or eliminated and movement and leakage of caustic bowel contents onto patient skin is also minimized. The outer sheath 18 includes a retention mechanism 22 configured to prevent movement of the outer sheath 18 within the patient. It should be noted that, if the inner cannula fails, it can be removed and nutrition can be delivered to the patient through the lumen in the outer sheath where the inner cannula is supposed to be, as an emergency backup.

As illustrated in FIG. 2, the retention mechanism 22 takes the form of a balloon 38. However, any retention mechanism known to or conceivable to one of skill in the art, such as an anchor or flange can be used. The outer sheath 18 also includes a stiffener 24 and distal weights 26. The stiffener 24 can take the form of a reinforcement of the outer sheath 18, such as with a shape memory metal like NiTiNol. In other embodiments, the stiffener 24 can take the form of a reinforcement, wrapping, or separate reinforcing piece disposed around the outer sheath 18. In still other embodiments, the stiffener can be integrated into the outer sheath, with a harder material used for that portion of the extrusion of the sheath. Fenestrations 28 defined by the outer sheath 18 and the inner cannula 20 allow for gastric port medications to be dispensed to the patient and to allow for gastric venting. The inner cannula 20 extends beyond a distal end 30 of the outer sheath 18.

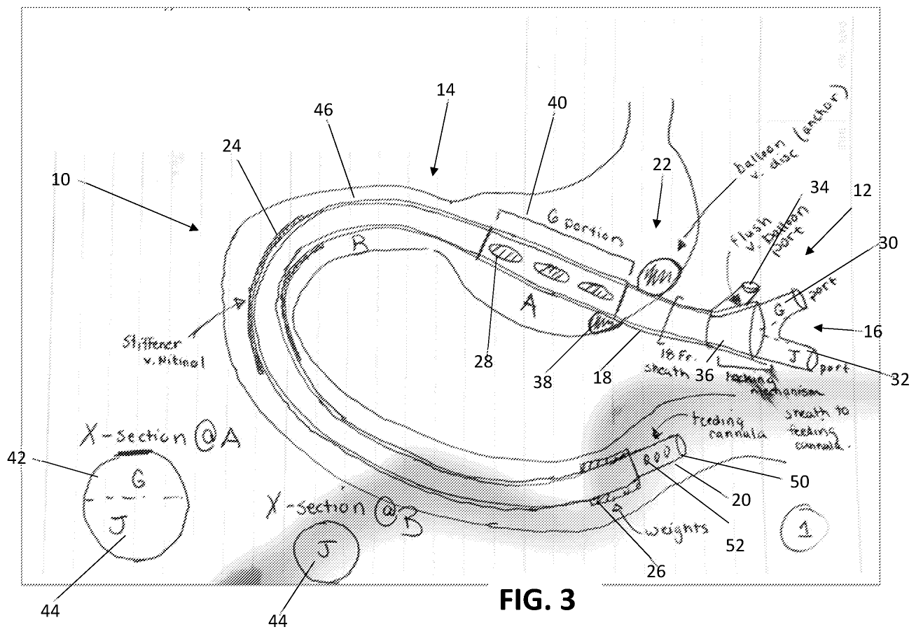

FIG. 3 illustrates a side view of a sleeved GJ tube of the present invention disposed in the intestinal tract. The sleeved GJ tube 10 includes external and internal portions 12, 14. The external portion 12 includes a two-port feeding hub 16. The inner portion 14 includes an outer sheath 18 and an inner cannula 20. While a two-port feeding hub 16 is shown here, it is to be understood that this is simply included as an example and is not meant to be considered limiting. Any feeding hub configuration known to or conceivable to one of skill in the art could be used. As illustrated in FIG. 3, the two-port feeding hub 16 includes a gastric port 30 and a jejeunal port 32. The two-port feeding hub 16 can also include a flush port and/or a balloon port 34. The external portion 12 also includes a locking mechanism 36. The locking mechanism 36 locks the outer sheath 18 to the inner cannula 20. The outer sheath 20 includes a retention mechanism 22 configured to prevent movement of the outer sheath 20 within the patient.

As illustrated in FIG. 3, the retention mechanism 22 takes the form of a balloon 38. The balloon 38 can be inflated with the balloon port 34 or via any other mechanism known to or conceivable to one of skill in the art, such as injection of a fluid into the balloon 38. The balloon port 38 can include a one way valve to prevent leakage of the inflation material (air/saline). However, any retention mechanism 22 known to or conceivable to one of skill in the art, such as an anchor or flange can be used. For the internal portion, FIG. 3 illustrates an 18 French outer sheath 18. Any size of outer sheath or inner cannula suitable for the patient and known to or conceivable to one of skill in the art can be used. The outer sheath 18 and the inner cannula 20 define fenestrations 28 to allow for gastric port medications to be dispensed to the patient from the gastric portion 40 of the sleeved GJ tube 10. The outer sheath 18 also includes a stiffener 24 and distal weights 26. The stiffener 24 can take the form of a reinforced area of the outer sheath 18, a wrapping with a shape memory material, such as NiTiNol, or another stiffener known to or conceivable by one of skill in the art.

Further as illustrated with respect to FIG. 3, the inner cannula 20 is disposed within a lumen defined by outer sheath 18. The outer sheath and the inner cannula 18, 20 define fenestrations 28, configured for delivery of medication or other substances along the gastric portion of the device. A cross-section of the inner cannula 20 taken along the gastric portion 40 of the sleeved GJ tube 10 includes a first lumen 42 and a second lumen 44. The first and second lumens 42 and 44 can take any configuration known to or conceivable to one of skill in the art. Distal to the gastric portion 40 of the sleeved GJ tube 10 is the jejunal or intestinal portion 46. A cross-section of the inner cannula 20 taken along the jejunal portion 46 includes only one lumen 44. The structure of the lumens of the inner cannula, is included herein with respect to FIG. 3, by way of example. This example is not meant to be considered limiting and any structural arrangement of the lumen(s) of the inner cannula 20 known to or conceivable to one of skill in the art is considered to be included herein. A distal end 50 of the inner cannula 20 extends beyond a distal end 30 of the outer sheath 18. The inner cannula 20 is slidably disposable within the lumen of the outer sheath 18. The inner cannula 20 can be locked in place via locking mechanism 36, such that the inner cannula 20 is immobile within the outer sheath 18, until it is to be removed and/or replaced. In some embodiments the inner cannula 20 can also define distal fenestrations 52.

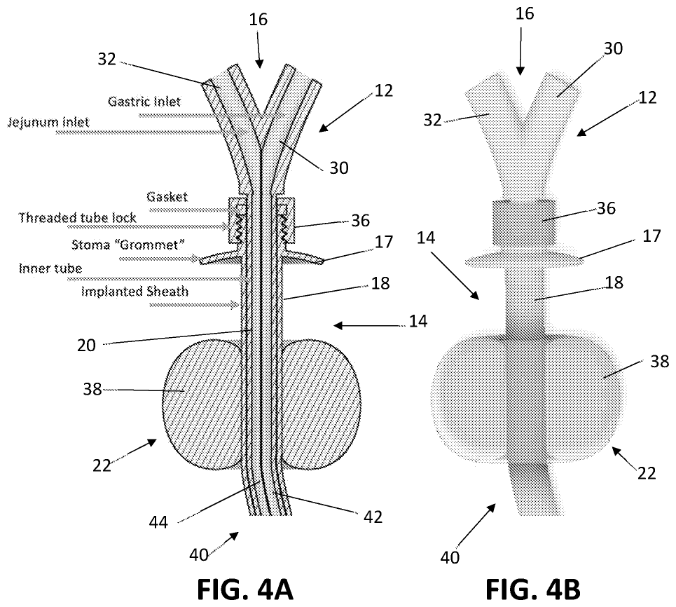

FIGS. 4A and 4B illustrate sectional and side views of the external portion and a proximal portion of the internal portion of the sleeved GJ tube, according to an embodiment of the present invention. The external portion 12 includes a two-port feeding hub 16. The inner portion 14 includes an outer sheath 18 and an inner cannula 20. While a two-port feeding hub 16 is shown here, it is to be understood that this is simply included as an example and is not meant to be considered limiting. Any feeding hub configuration known to or conceivable to one of skill in the art could be used. As illustrated in FIGS. 4A and 4B, the two-port feeding hub 16 includes a gastric port 30 and a jejeunal port 32. The external portion 12 also includes a locking mechanism 36. The locking mechanism 36 locks the outer sheath 18 to the inner cannula 20. As illustrated in FIGS. 4A and 4B, the locking mechanism 36 takes the form of a threaded tube lock. The threaded tube lock is included by way of example and any suitable locking mechanism known to or conceivable to one of skill in the art could also be used, including a tab and groove lock or a pressure fitting. A flange 17 disposed on the external portion 12 covers the stoma of the patient. As illustrated in FIGS. 4A and 4B the flange 17 takes the form of a grommet that covers the stoma. It should be noted that any configuration or design of flange 17 known to or conceivable to one of skill in the art could also be used.

The internal portion 14 of the outer sheath 20 includes a retention mechanism 22 configured to prevent movement of the outer sheath 20 within the patient. In FIGS. 4A and 4B, the retention mechanism 22 takes the form of a balloon 38. However, any retention mechanism 22 known to or conceivable to one of skill in the art, such as an anchor or flange can be used. The balloon 38 can be inflated with the balloon port 34 or via any other mechanism known to or conceivable to one of skill in the art, such as injecting a fluid into the balloon. A cross-section of the inner cannula 20 taken along the gastric portion 40 of the sleeved GJ tube 10 includes a first lumen 42 and a second lumen 44. The first and second lumens 42 and 44 can take any configuration known to or conceivable to one of skill in the art.

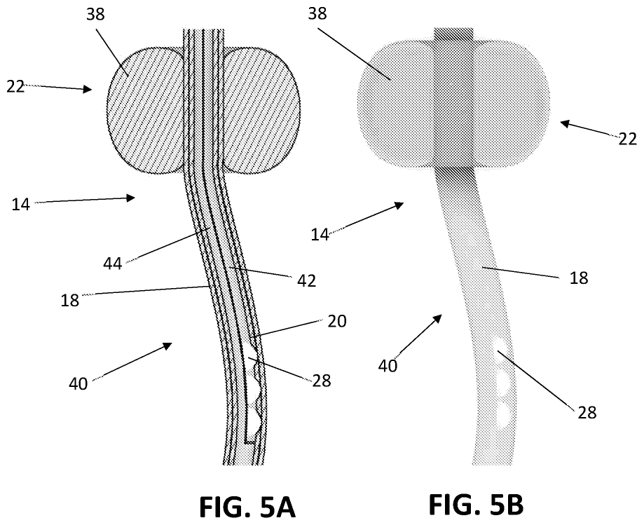

FIGS. 5A and 5B illustrate sectional and side views of the balloon retention mechanism and the fenestrations of the outer sheath and inner cannula, according to an embodiment of the present invention. FIGS. 5A and 5B illustrate the internal portion 14 of the outer sheath 18 and the inner cannula 20. The inner cannula 20 is slidably disposed within the outer sheath 18, such that it can be removed and/or replaced. The outer sheath 18 includes retention mechanism 22. As illustrated in FIGS. 5A and 5B, the retention mechanism 22 takes the form of a balloon 38. However, any retention mechanism 22 known to or conceivable to one of skill in the art, such as an anchor or flange can be used. For the internal portion, the outer sheath 18 and the inner cannula 20 also define fenestrations 28 to allow for gastric port medications to be dispensed to the patient from the gastric portion 40 of the sleeved GJ tube 10. As illustrated in FIG. 5A, the cross-section of the inner cannula 20 taken along the gastric portion 40 of the sleeved GJ tube 10 includes a first lumen 42 and a second lumen 44. The first and second lumens 42, 44 can take any configuration known to or conceivable to one of skill in the art. As illustrated in FIG. 5A, the second lumen 42 is used for delivering medication to the stomach of the patient. The second lumen 42 terminates just below the fenestrations, as illustrated in FIG. 5A, however the first and second lumens 42, 44 can have any configuration, as would be known to one of skill in the art.

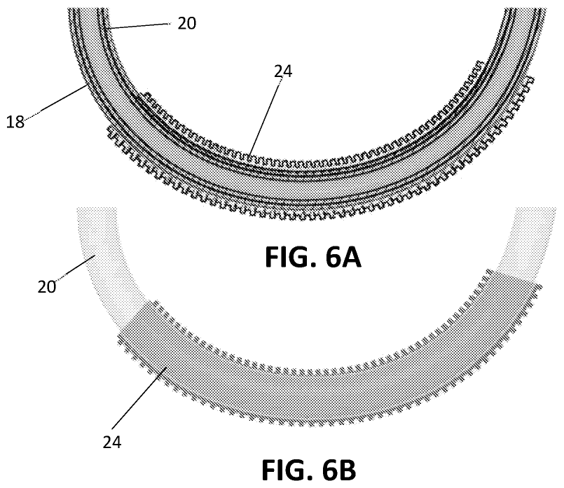

FIGS. 6A and 6B illustrate sectional and side views of the stiffener of the outer sheath, according to an embodiment of the present invention. The stiffener 24 can take the form of a reinforced area of the outer sheath 18, a wrapping with a shape memory material, such as NiTiNol, or another stiffener known to or conceivable by one of skill in the art. Alternately, a stiffening device could be positioned within the outer sheath 18 and or the inner cannula 20 of the device.

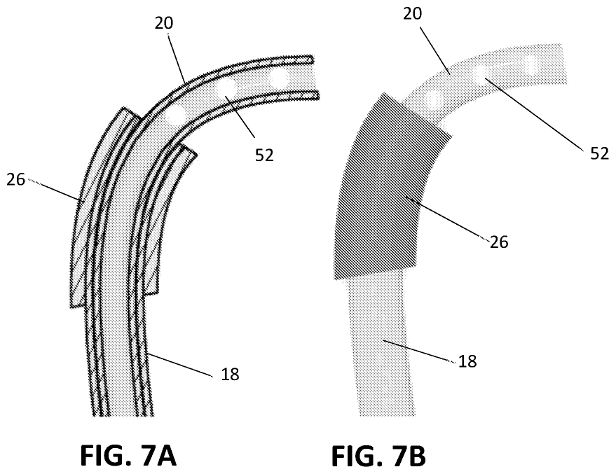

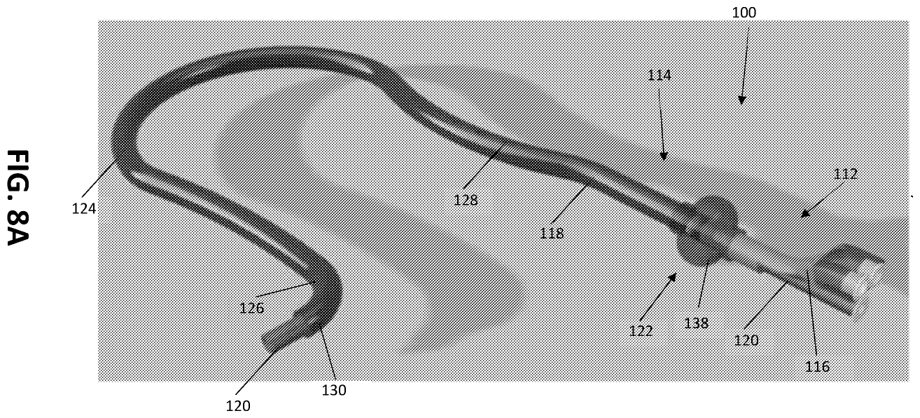

FIGS. 7A and 7B illustrate sectional and side views of the distal end of the outer sheath along with the distal end of the inner cannula, according to an embodiment of the present invention. As illustrated in FIGS. 7A and 7B, a distal end 50 of the inner cannula 20 extends beyond a distal end 30 of the outer sheath 18. The inner cannula 20 is slidably disposable within the lumen of the outer sheath 18 for removal or replacement. In some embodiments, the inner cannula 20 can also define distal fenestrations 52. FIGS. 7A and 7B also illustrate distal weights 26. The distal weights 26 are disposed at a distal end of the outer sheath 20.

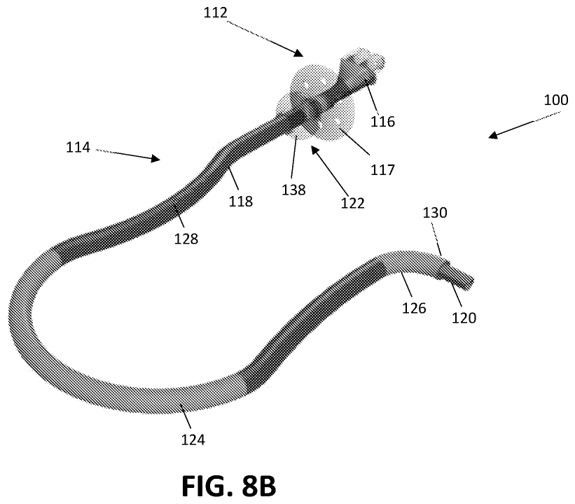

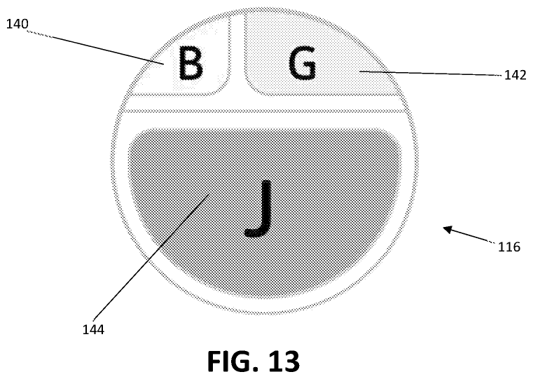

FIGS. 8A and 8B illustrate perspective views of a sleeved GJ tube, according to an embodiment of the present invention. The sleeved GJ tube 100 includes external and internal portions 112, 114. The external portion 112 includes a hub 116. The hub 116 can include any number of ports known to or conceivable to one of skill in the art. In some embodiments, the hub 116 can include three ports: a balloon port, a gastric port, and a jejunal port. In other embodiments, the hub 116 can include four ports: a balloon port, a gastric port, a jejunal port, and a lubrication port. As illustrated in FIG. 8B, a flange 117 is disposed on the external portion 112, and covers the stoma of the patient. The ports are described in more detail with respect to FIGS. 13A and 13B herein. The internal portion 114 includes an outer sheath 118 and an inner cannula 120. The inner cannula 120 in many embodiments takes the form of a feeding cannula. The inner cannula 120 can be configured to lock with the outer sheath 118 such that unwanted movement between the two components is minimized or eliminated. The outer sheath 118 includes a retention mechanism 122 configured to prevent movement of the outer sheath 118 within the patient. It should be noted that, if the inner cannula fails, it can be removed and nutrition can be delivered to the patient through the lumen in the outer sheath where the inner cannula is supposed to be, as an emergency backup.

As illustrated in FIGS. 8A and 8B, the retention mechanism 122 takes the form of a balloon 138. However, any retention mechanism known to or conceivable to one of skill in the art, such as an anchor or flange can be used. The outer sheath 118 also includes a stiffener 124 and distal weights 126. The stiffener 124 can take the form of a reinforcement of the outer sheath 118, such as with a shape memory metal like NiTiNol. In other embodiments, the stiffener 124 can take the form of a reinforcement, wrapping, or separate reinforcing piece disposed around the outer sheath 118. Fenestrations 128 defined by the outer sheath 118 and the inner cannula 120 allow for gastric port medications to be dispensed to the patient. The inner cannula 120 extends beyond a distal end 130 of the outer sheath 118.

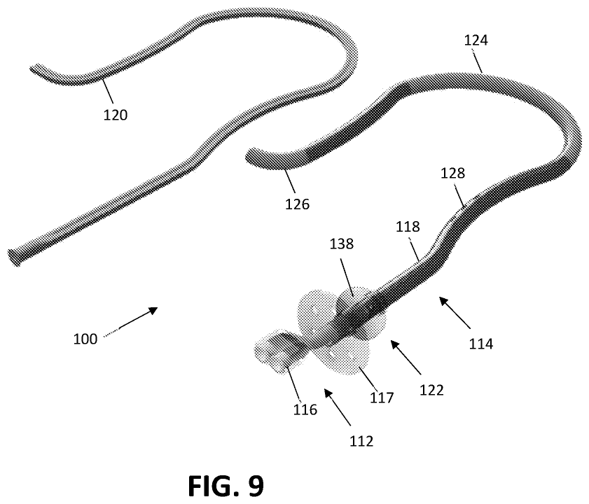

FIG. 9 illustrates a semi-exploded view of a sleeved GJ tube, according to an embodiment of the present invention. The sleeved GJ tube 100 includes external and internal portions 112, 114. The external portion 112 includes a hub 116. The hub 116 can include any number of ports known to or conceivable to one of skill in the art. In some embodiments, the hub 116 can include three ports: a balloon port, a gastric port, and a jejunal port. In other embodiments, the hub 116 can include four ports: a balloon port, a gastric port, a jejunal port, and a lubrication port. A flange 117 is disposed on the external portion 112, and covers the stoma of the patient. The ports are described in more detail with respect to FIGS. 13A and 13B herein. The internal portion 114 includes an outer sheath 118 and an inner cannula 120. The inner cannula 120 in many embodiments takes the form of a feeding cannula. The inner cannula 120 can be configured to lock with the outer sheath 118 such that unwanted movement between the two components is minimized or eliminated. The outer sheath 118 includes a retention mechanism 122 configured to prevent movement of the outer sheath 118 within the patient. As shown in FIG. 9, the retention mechanism 122 takes the form of a balloon 138. However, any retention mechanism known to or conceivable to one of skill in the art, such as an anchor or flange can be used. The outer sheath 118 also includes a stiffener 124 and distal weights 126. The stiffener 124 can take the form of a reinforcement of the outer sheath 118, such as with a shape memory metal like NiTiNol. In other embodiments, the stiffener 124 can take the form of a reinforcement, wrapping, or separate reinforcing piece disposed around the outer sheath 118. Fenestrations 128 defined by the outer sheath 118 and the inner cannula 120 allow for gastric port medications to be dispensed to the patient. The inner cannula 120 extends beyond a distal end 130 of the outer sheath 118.

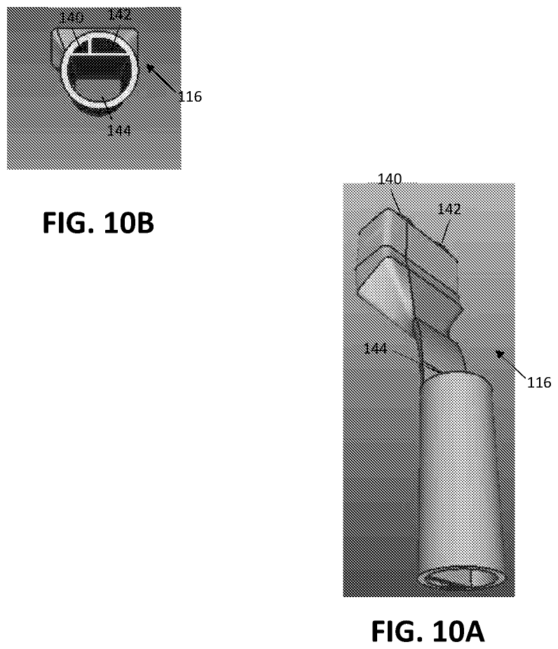

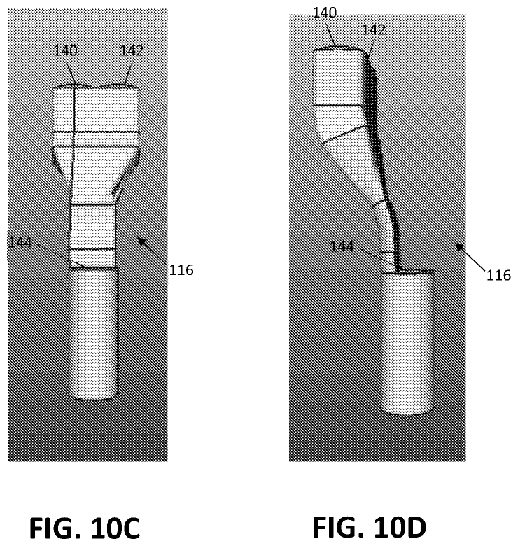

FIGS. 10A-10D illustrate views of a hub of a sleeved GJ tube, according to an embodiment of the present invention. The hub 116 can include any number of ports known to or conceivable to one of skill in the art. In some embodiments, the hub 116 can include three ports: a balloon port 140, a gastric port 142, and a jejunal port 144. In other embodiments, the hub 116 can include four ports: a balloon port, a gastric port, a jejunal port, and a lubrication port.

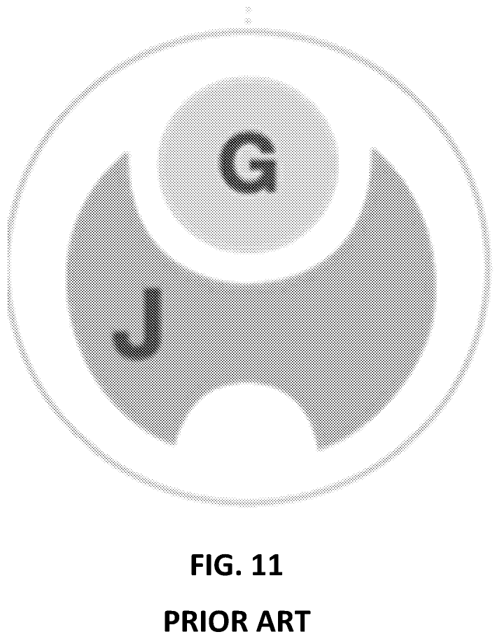

FIG. 11 illustrates a sectional view of a lumen arrangement of a device according to the prior art. The prior art design for the hub and lumens includes two to three ports: a gastric port and a jejunal port, and in some cases a balloon port if that is the retention mechanism being used. Current designs have many sharp corners in the jejunal portion, where food accumulates and causes clogs, as illustrated in FIG. 11.

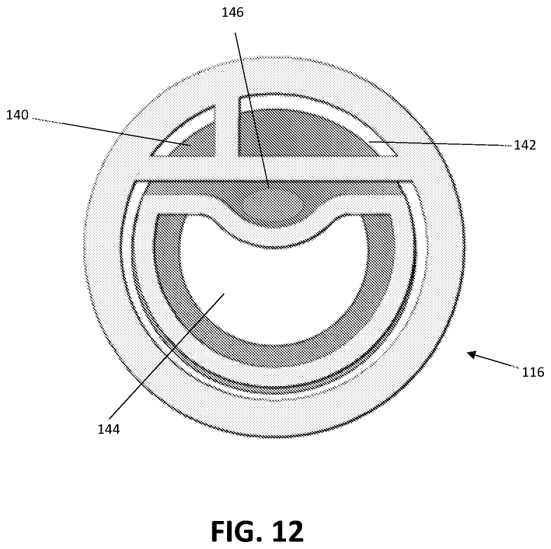

FIGS. 12 and 13 illustrate sectional views of a lumen arrangement of a sleeved GJ tube, according to an embodiment of the present invention. As illustrated in FIGS. 12 and 13 the port arrangement of the hub 116 of the present invention reduces the number of sharp corners in the jejunal port 144. By placing the balloon port 140 and gastric ports 142 next to each other, the lumen sizes for all three ports is bigger, which will aid in nutrition delivery and reduce clogging. It should also be noted that all of the cross sectional areas of the lumens of the present design are equal to or larger than those in the prior art GJ tubes. Therefore, performance is increased and no decreases in performance are seen. FIG. 12 illustrates a lubrication port 146, also disposed in the port arrangement of the hub 116. Oil-based lubricants and silicone coatings can also be used. Lubricant can be delivered through the hub or through an infusion wire that snakes down a space between the inner cannula and the outer sheath. The curved ‘dent’ in the inner cannula is designed to allow lubricant to flow and/or an infusion wire to travel, as illustrated in FIG. 12.

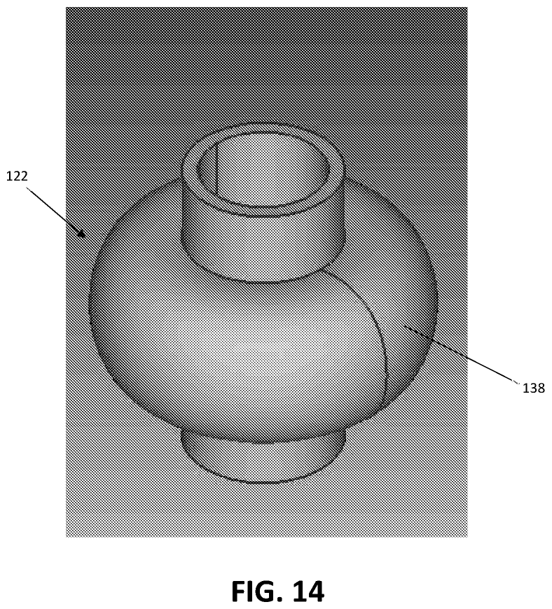

FIG. 14 illustrates a perspective view of a balloon retention mechanism of a sleeved GJ tube, according to an embodiment of the present invention. As illustrated in FIGS. 5A and 5B, the retention mechanism 122 takes the form of a balloon 138. However, any retention mechanism 122 known to or conceivable to one of skill in the art, such as an anchor or flange can be used. The balloon 138 is insufflated via the balloon port described herein, with respect to FIGS. 12, 13A, and 13B.

With respect to materials for the sheath and cannula, multiple elastomers were tested for their stiffness, biocompatibility, and frictional properties and multiple lubricants to reduce the friction between tubes. Silicone has the best combined stiffness, biocompatibility, and coefficient of friction. MED6670 (dry) and MS3130 (wet) lubricants provide the best reduction in friction between the tubing. Oil-based lubricants and silicone coatings can also be used. Lubricant can be delivered through the hub or through an infusion wire that snakes down a space between the inner cannula and the outer sheath. Degradation was tested in harsh acidic environments that mimic the abdomen. Complex intestinal curves were 3D printed to confirm that the selected material and lubricants will easily slide in and out. It should be noted that it is not preferable to use stock silicone tubing, in order to maximize the lumen size, reduce the chances of clogging, and add a port for lubrication. Custom silicone profiles associated with certain embodiments of the present invention can only be made by a commercial silicone extruder.

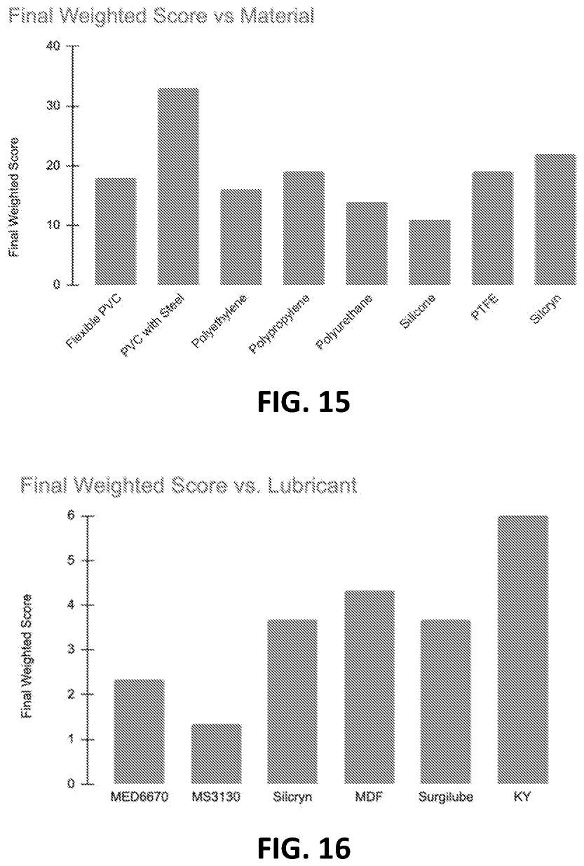

FIGS. 15 and 16 illustrate graphical views for the materials tested and lubricants tested, according to embodiments of the present invention. FIG. 15 shows the final weighted scores of all materials tested for stiffness, biocompatibility, and frictional properties. FIG. 16 shows final weighted scores of all lubricants tested reduction in friction through curved and acidic environment testing. For both FIGS. 15 and 16, lower scores indicate better performance. Table 3 lists exemplary possible materials that could be used to form the present invention, and Table 4 lists exemplary possible lubricants that could be used in conjunction with the present invention.

TABLE 3
Material Rankings for Various Tests

| Bend Radius | COF | Overall |
|---|---|---|
| Silicone | Semi-flexible PVC w/Steel | Silicone |
| Polyurethane (Versilon) | Polyethylene | Polyurethane (Versilon) |
| Flexible PVC (Tygon) | PTFE | Polypropylene/EPDM Blend |
| Polypropylene/EPDM Blend | Polypropylene/EPDM Blend | Flexible PVC (Tygon) |
| Polyethylene | Polyurethane (Versilon) | Polyethylene |
| Semi-flexible PVC w/Steel | Silicone | Semi-flexible PVC w/Steel |
| Polyethylene with aluminum | Flexible PVC (Tygon) | PTFE |
| reinforcement | | |
| PTFE | Polyethylene with aluminum | Polyethylene with aluminum |
| | reinforcement | reinforcement |

TABLE 4
Lubricants

| Dry Test | Acid Test | Curve Test | Overall |
|---|---|---|---|
| MED6670 | MS3130 | MS3130 | MS3130 |
| MS3130 | Surgilube | MED6670 | MED6670 |
| Silcryn | MDF | Silcryn | Silcryn/Surgilube |
| MDF | MED6670 | Surgilube | Silcryn/Surgilube |
| Surgilube | Silcryn | MDF | MDF |
| KY | KY | KY | KY |

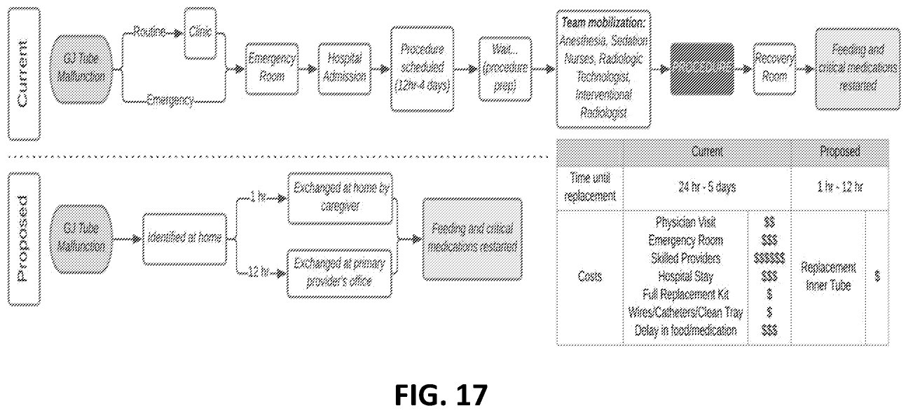

FIG. 17 illustrates a flow diagram of a patient care flow according to an embodiment of the present invention. Currently, when a patient's GJ tube malfunctions, they can either visit an outpatient clinic or go directly to the emergency room, as illustrated in the top half of FIG. 17. Through either route, the patient is then admitted to the hospital and a procedure is scheduled where the wait can range from 12 hours to 4 days. After the patient is prepped, an expensive and expansive team of anesthesiologists, technologists, nurses, and procedural physicians (i.e., interventional radiologists) must be mobilized to perform the procedure. Specialized fluoroscopy suites with X-ray must be used for the replacement. The patient and their insurance face significant financial costs for the emergency room visit, hospital stay, intravenous food and medication, team of specialists, procedure, and recovery. There is also a significant risk to the patient's health as they are without necessary enteral food and medication until the procedure is complete and are exposed to radiation. At the absolute minimum, this process takes 24 hours but generally takes multiple days. Finally, the patient's at-home caregivers are negatively impacted through the urgent hospital visits which can cause numerous work and lifestyle disruptions.

The present invention aims to simplify the entire replacement workflow and make life easier for patients, families, and providers, as illustrated in the bottom half of FIG. 17. With the device of the present invention, when a GJ tube malfunctions, the patient can have it replaced at home in less than 1 hour if the caregiver is comfortable. Otherwise, they can visit any primary care provider's office and have the tube replaced within hours. Critical food and medication can be restarted immediately, and the patient faces very little delay. Furthermore, the expensive care team, specialized procedure room, and extended recovery from an invasive procedure are completely removed from the process. Simply, the device of the present invention creates up to a 10× reduction in time to replacement and a 2-3× reduction in hospital visits and all the associated costs (both financial and health-related).

The present invention can take the form of a kit. The main kit can include 2 parts: a flexible, replaceable inner feeding tube and a long-lasting outer sheath with stiffener, retention mechanism, distal anchor, and feeding hub. A separate kit can include a replacement inner tube. It is during scheduled and non-scheduled GJ tube replacements that the present invention significantly alters workflow leading to decreased time to replacement, decreased risk to the patient, and decreased cost for the patient, their caregivers, and health systems by ‘taking the procedure home’, as illustrated in FIG. 17.

For health systems, by taking the GJ tube replacement procedure to the bedside, significant Interventionalist time is freed up. Conservatively, the present invention could reduce the number of hospital visits by 50%. At Johns Hopkins alone this would free up 9 weeks or 20% of a physician's time to perform higher-value procedures. Scaled across thousands of hospitals, a staggering amount of physician time is freed up, patient hospital visits are reduced, and revenue per year per patient for feeding tube supplies is increased.

GJ tubes are recommended to be replaced every 6-12 months, but they often fail after 3-4 months because of clogs, displacements, and/or leaks. By moving tube replacement to the bedside, patients can preventatively replace their inner tube every 2 months. Further, performing the procedure at the bedside does not increase infection risk. GJ tube replacement is currently performed in procedure suites due to the need for fluoroscopic imaging and wires/catheters for exchanges of current systems. These procedures are considered “clean” and not sterile. Exchanging feeding tubes involves the patient's existing, established track, and endothelialized connection between the stomach and the skin. Of note, the stomach is inherently “dirty” making sterility both impossible and not a concern.

Although the present invention has been described in connection with preferred embodiments thereof, it will be appreciated by those skilled in the art that additions, deletions, modifications, and substitutions not specifically described may be made without departing from the spirit and scope of the invention as defined in the appended claims. It should be noted that while the present invention is described in part with respect to pediatric applications it is to be understood that the device can be used with patients of any age that require a GJ tube.

1. A sleeved gastrojejunostomy (GJ) tube device comprising:
an outer sheath having a proximal end and a distal end, wherein an outer wall of the outer sheath defines an inner lumen that extends from the proximal end of the outer sheath to the distal end of the outer sheath;
an inner cannula having a proximal end and a distal end, wherein an outer wall of the inner cannula defines an inner lumen that extends from the proximal end of the inner cannula to the distal end of the inner cannula, wherein the inner cannula is removably disposed within the inner lumen of the outer sheath;
a hub disposed at a proximal end of the device, wherein the hub comprises ports to provide access to the gastric and jejunal spaces.

2. The device of claim 1 further comprising a retention mechanism to hold the GJ tube device in place.

3. The device of claim 2 wherein the retention mechanism comprises a balloon.

4. The device of claim 1 wherein the outer sheath and the inner cannula define fenestrations for delivering medication or other substances to a patient.

5. The device of claim 1 further comprising a stiffener.

6. The device of claim 1 further comprising distal weights positioned on a distal end of the outer sheath.

7. The device of claim 1 further comprising a locking mechanism to releasably couple the inner cannula to the outer sheath.

8. The device of claim 1 wherein the locking mechanism comprises a threaded tube lock.

9. The device of claim 1 further comprising a grommet to cover a stoma of the patient.

10. The device of claim 1 wherein at least a portion of the inner cannula defines a first lumen and a second lumen.

11. The device of claim 3 wherein the hub is further configured with a balloon port to allow for insufflation of the balloon.

12. The device of claim 1 wherein the hub is further configured with a lubrication port to provide lubrication between the outer sheath and the inner cannula.

13. The device of claim 12 further comprising at least one selected from a group consisting of cured silicone coating, oil-based lubricants, MED6670, and MS3130 lubricants.

14. The device of claim 1 wherein the outer sheath and the inner cannula are formed from silicone.

15. The device of claim 5 wherein the stiffener is formed from one selected from a group consisting of a shape memory metal, a hardened strip of plastic, a hardened strip of rubber, and a hardened integral portion of the outer sheath.

16. The device of claim 15 wherein the stiffener is formed from NiTiNol.

17. The device of claim 14 wherein the device is formed with one selected from a group consisting of a silicone extruder, injection molding, and casting.

18. A kit comprising the device of claim 1.

19. The kit of claim 19 further comprising a lubricant.

20. A second kit comprising at least one replacement inner cannula wherein the replacement inner cannula is configured to replace the inner cannula provided in the kit of claim 18.
